(19) United States
(12) Patent Application Publication
(10) Pub. No.: US 2010/0217399 A1
(43) Pub. Date: Aug. 26, 2010

(54) BASE PLATE SYSTEM FOR SHOULDER ARTHROPLASTY AND METHOD OF USING THE SAME

(76) Inventor: Gordon I. Groh, Ashville, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/710,084

(22) Filed: Feb. 22, 2010

Related U.S. Application Data
(60) Provisional application No. 61/154,429, filed on Feb. 22, 2009.

(51) Int. Cl. A61F 2/40; A61B 17/56

(52) U.S. Cl. 623/19.11; 606/96

(57) ABSTRACT

Embodiments of the present invention provide systems and methods for use in a replacing a joint having a prepared bone surface. A base plate system according to one embodiment includes a base plate comprising a plurality of locking screw holes defined therethrough and first and second opposing surfaces, wherein the second surface is configured to be positioned adjacent to the prepared bone surface. The system also includes a central peg extending outwardly from the second surface of the base plate and configured to engage the prepared bone surface and a plurality of locking screws configured to be inserted within a respective locking screw hole and engage the prepared bone surface. In addition, the system includes a plurality of locking washers disposed within a respective locking screw hole and configured to receive a respective locking screw and lock the locking screw at a desired angle with respect to the base plate.

### CROSS-REFERENCE TO RELATED APPLICATIONS

The present application claims priority from U.S. Provisional Application No. 61/154,429 filed Feb. 22, 2009, the contents of which are incorporated herein by reference.

### FIELD OF INVENTION

Various embodiments of the present invention relate to shoulder arthroplasty and, in particular, to a base plate system for use in reverse arthroplasty and total arthroplasty procedures.

### BACKGROUND OF THE INVENTION

Reverse shoulder arthroplasty is indicated for rotator cuff deficient arthritis of the shoulder. In these patients, the rotator cuff which stabilizes a conventional arthroplasty is deficient. By reversing the ball and socket configuration, a stable construct may be implanted which allows the intact deltoid muscle to contract under an improved lever arm. This reversal also allows for improved elevation of the shoulder.

Reverse shoulder arthroplasty continues to remain challenging. There are separate issues both in implantation of devices and dealing with bone deficiency. In this regard, current designs for base plates for shoulder arthroplasty do not allow for locking screws that can be redirected without damaging the locking mechanism. In addition, there further exists a need for a more versatile central post of the base plate to also improve fixation to bone.

In reverse shoulder arthroplasty or total shoulder arthroplasty, bone loss affecting the glenoid is not unusual. The current methods to deal with bone loss involve either eccentric reaming of the glenoid or bone grafting the defect. Eccentric reaming remains the easiest mechanism to deal with bone loss, but this technique has its limits. First, reaming can only typically correct defects of 10 degrees of version of the glenoid. Eccentric reaming above these parameters involves removal of over 5 mm of bone stock. Reaming above this level causes marked narrowing of the glenoid due to the trumpet configuration of the scapular neck. If the glenoid face is over narrowed via reaming, adequate seating and screw/peg fixation of an implant is very difficult if not impossible.

The second option to deal with bone loss has been bone grafting. Unfortunately, this technique is extremely demanding and time consuming. To date there exist no jigs or preformed grafts to make this procedure easier. Further, the technique requires a graft which may not be available as a native bone, necessitating the use of an allograft. Finally, graft to native bone healing is not assured and may lead to early or late failure of the prosthesis.

Thus, there remains a need in the art for an improved system and method for use with shoulder arthoplasty. In particular, there is a need for a system that more effectively fixates the base plate to the bone. In addition, there is a need for a system that more effectively addresses bone loss in any portion of the glenoid.

### SUMMARY OF THE INVENTION

The above and other needs may be met by embodiments of the present invention which, in one embodiment, provides a base plate system for use in a replacing a joint having a prepared bone surface, such as the glenoid. A base plate system according to one embodiment includes a base plate comprising a plurality of locking screw holes defined therethrough and first and second opposing surfaces, wherein at least a portion of the second surface is configured to be positioned adjacent to the prepared bone surface. The system also includes a central peg extending outwardly from the second surface of the base plate and configured to at least partially engage the prepared bone surface, as well as a plurality of locking screws configured to be inserted within a respective locking screw hole and engage the prepared bone surface. In addition, the system includes a plurality of locking washers disposed within a respective locking screw hole and configured to receive a respective locking screw and lock the locking screw at a desired angle with respect to the base plate.

According to aspects of the system, each of the plurality of locking screw holes includes an annular groove for receiving a respective locking washer therein. A diameter and a thickness of each of the annular grooves may be larger than an outer diameter and a thickness, respectively, of each of the plurality of locking washers. Moreover, each of the locking washers may be radially resilient and configured to adjust in diameter in response to engagement with a respective locking screw. A diameter of each of the locking screw holes may be larger than an inner diameter of each of the plurality of locking washers.

In additional aspects, the central peg is integrally formed with the base plate. Alternatively, the central peg is configured to be secured to the base plate and is variable in length. The central peg may include a peg hole configured to receive a peg locking screw therethrough for engaging the bone. Or, the peg locking screw may be integrally formed with the central peg and the base plate. In addition, the system may include a locking washer positioned within the peg hole and configured to lock the peg locking screw at a desired angle with respect to the base plate. In one aspect, the peg locking screw has a length of about 10 to 45 mm and a diameter of about 6 to 7 mm, while the central peg has a length of about 10 to 40 mm. At least a portion of the second surface of the base plate and the central peg may be coated with a bioingrowth material. The plurality of locking screws may have a length of about 5 to 45 mm and a diameter of about 4 to 6 mm. The locking washers may be configured to lock the locking screw at an angle of up to about 15 degrees in any direction with respect to the base plate. In another aspect, the system further includes a drill guide configured to engage one of the locking washers and a drill bit configured to be inserted through the drill guide and drill a hole through the prepared bone surface for receiving a respective locking screw.

In an additional embodiment, the system includes an augmentation device configured to be secured to the base plate and positioned adjacent to the prepared bone surface to accommodate for bone loss of the joint. The augmentation device may include a plurality of screw holes configured to align with a plurality of screw holes defined in the base plate, wherein the augmentation device is configured to be secured to the base plate by inserting a plurality of respective screws through the screw holes defined in the augmentation device and the base plate. The augmentation device may have a wedge or rectangular cross-sectional shape, while a maximum thickness of the augmentation device may be about 5 to 40 mm. Moreover, at least a portion of the augmentation device may be coated with, or formed of, a bioingrowth material. The augmentation device may include a pair of opposing surfaces and an inner edge and an outer edge extending therebetween, wherein a substantial portion of the outer edge corresponds to an outer edge of the base plate. The inner edge of the augmentation device may include a plurality of contours configured to at least partially conform to the plurality of locking screws and the central peg. In addition, the augmentation device may include at least one hole configured to align with a respective one of the plurality of locking screw holes and thereby receive a locking screw therethrough.

Another embodiment of the present invention is directed to a method for installing a bone plate system for replacing a joint having a prepared bone surface, such a prepared bone surface of the glenoid. The method includes positioning a base plate adjacent to the prepared bone surface such that a central peg extending outwardly from the base plate engages the prepared bone surface, wherein the base plate includes a plurality of locking screw holes defined therethrough and a plurality of locking washers disposed within a respective locking screw hole. The method further includes inserting a plurality of locking screws through a respective locking screw hole and locking washer such that the locking screws are fixed therein and extend through the prepared bone surface at a desired angle with respect to the base plate.

According to one aspect of the method, the inserting step includes inserting the plurality of locking screws through a respective locking screw hole and locking washer such that the locking screws are fixed at an angle of up to about 15 degrees in any direction with respect to the base plate. The method may also include inserting a peg locking screw through a peg hole defined in the central peg such that the peg locking screw engages the prepared bone surface. The inserting step may include inserting the peg locking screw through a locking washer positioned within the peg hole to lock the peg locking screw at a desired angle with respect to the base plate. Furthermore, the method may include adjusting a length of the central peg. The method may include attaching a drill guide to one of the plurality of locking washers and inserting a drill bit through the drill guide to drill a hole through the prepared bone surface for receiving a respective locking screw. Moreover, the method may include securing an augmentation device to the base plate and positioning the augmentation device adjacent to the prepared bone surface to accommodate for bone loss of the joint. The securing step may include positioning a plurality of contours defined in the augmentation device at least partially around the locking screws and the central peg.

### BRIEF DESCRIPTION OF THE DRAWINGS

Various embodiments of the invention will be better understood by reference to the Detailed Description of Various Embodiments of the Invention when taken together with the attached drawings, wherein:

FIG. 1 is a perspective view of a base plate system according to one embodiment of the present invention;

FIG. 1A is a partial cross-sectional view of a base plate taken through a locking screw hole and a locking washer according to one embodiment of the present invention;

FIG. 1B is a perspective view of a locking washer according an embodiment of the present invention;

FIG. 2 is a perspective view of an augmentation device according to one embodiment of the present invention; and

FIG. 3 is a perspective view of an augmentation device according to another embodiment of the present invention.

### DETAILED DESCRIPTION OF VARIOUS EMBODIMENTS OF THE INVENTION

Various embodiments of the present invention now will be described more fully hereinafter with reference to the accompanying drawings, in which some, but not all embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like numbers refer to like elements throughout. The singular forms “a,” “an,” and “the” include plural referents unless the context clearly dictates otherwise.

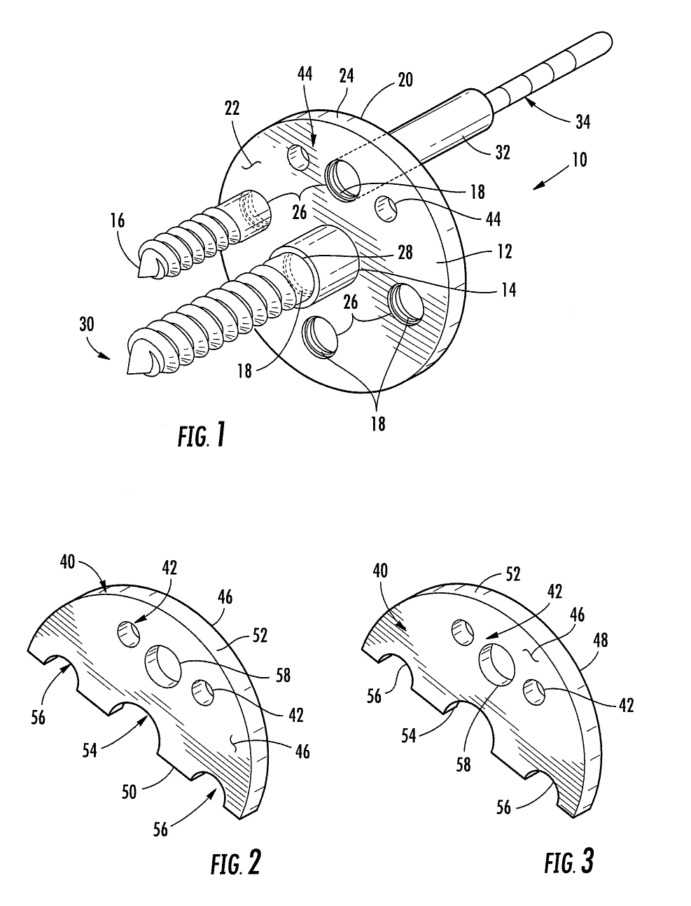

As shown generally in FIG. 1, embodiments of the present invention are directed to a base plate system 10 that generally includes a base plate 12, a central peg 14 extending from the base plate, a plurality of locking screws 16 configured to engage the bone, and a plurality of locking washers 18 configured to fix the locking screws with respect to the base plate at a desired angle. As explained in further detail below, the system 10 is capable of being positioned adjacent to a prepared bone surface, such as a prepared bone surface of the glenoid, for replacing a joint and being secured thereto. Although the embodiments are discussed in conjunction for use with total and reverse shoulder arthroplasty, it is understood that the system 10 may be adapted for use with other joints in accordance with additional embodiments of the present invention. For example, the system 10 may be modified for use with joints such as the wrist, ankle, or acetubulum where there is a conforming surface or otherwise a bone that is capable of being prepared to accommodate the base plate system 10.

In relation to a reverse shoulder arthroplasty, it is understood that a variety of components may be used in conjunction with the base plate system 10. For example, the base plate system 10 may be coupled to a glenoid head or glenosphere for articulating with respect to a humeral component, including a humeral socket and stem. Moreover, although the term “base plate” is used herein, it is understood that “base plate” is not meant to be limiting and may refer generally to any number of terms such as, for example, a backing plate, metaglene, glenoid plate, or glenoid component.

According to one embodiment, the base plate 12 includes first and second opposing surfaces 20, 22 and an outer surface 24 extending therebetween. At least a portion of the second surface 22 is configured to be positioned adjacent to a prepared bone surface. The second surface 22 may be substantially planar or have a convex or other outwardly extending surface for conforming to the prepared bone surface. Similarly, the first surface 20 may be substantially planar or have a concave or other inwardly extending surface. Although not shown, the first surface 20 may include a Morse taper or other engagement mechanism for engaging a glenoid head as known to those of ordinary skill in the art. The base plate 12 is typically circular in shape but may be other shapes such as, for example, oval, oblong, or rectangular if desired. In one embodiment, a conventional base plate may be modified to include the features of base plate 12, such as base plates disclosed by U.S. Pat. No. 6,679,916 to Frankle et al., U.S. Pat. No. 6,790,234 to Frankle, and U.S. Patent Appl. Publ. No. 2007/0244563 to Roche et al., each of which is incorporated by reference in its entirety herein.

Moreover, the base plate 12 comprises a plurality of peripheral locking screw holes 26 defined between the first and second opposing surfaces 20, 22 that are configured to receive respective peripheral locking screws 16, as explained in further detail below. The base plate 12 shown in FIG. 1 includes four locking screw holes 26 equidistantly spaced apart from one another about the central peg 14, although it is understood that various numbers and locations of locking screw holes may be provided if desired. In addition, the locking screws 16 may extend at any desired angle with respect to the base plate 12, such as parallel to one another, divergent or convergent with respect to one another, or at various oblique angles. Although the locking screws 16 may be various sizes and configurations, the locking screws are cancellous screws having a diameter of about 4-6 mm (e.g., 5 mm) and a length of about 5-45 mm, according to one embodiment of the present invention. Moreover, at least a portion of the second surface 22 may include a bioingrowth material, such a porous coating or hydroxyapatite, for facilitating bone in-growth.

The base plate 12 also includes a central peg 14 extending outwardly from the second surface 22 thereof and may be configured to at least partially engage the prepared bone surface. Depending on the size and configuration of the central peg 14, as well as the amount of bone loss, the central peg 14 may engage the prepared bone surface and underlying bone. The central peg 14 may be integrally formed from the same piece of material as the base plate 12 or may be independent of the base plate and attached thereto. Thus, in one embodiment, the base plate 12 is configured to receive the central peg 14 such that the central peg is variable in length. The central peg 14 may be secured to the base plate 12 using various techniques such as by fastening into the base plate or via a Morse taper. The central peg 14 may include a peg hole 28 defined therethrough that is sized and configured to receive a locking peg screw 30 that is configured to engage the bone. The peg locking screw 30 may be fixed in a desired orientation or may employ a locking washer 18 as described below for facilitating variability of the orientation of the peg locking screw. Furthermore, in another embodiment, the peg locking screw 30 may be integrally formed with the base plate 12. Thus, both the central peg 14 and peg locking screw 30 may be integrally formed with the base plate 12. Although the central peg 14 may be various sizes and configurations, in one embodiment, the central peg 14 is cylindrical in shape and is about 10-40 mm in length, while the peg locking screw is a cancellous screw of about 6-7 mm in diameter (e.g., 6.5 mm) and about 10-45 mm in length. According to one aspect, the central peg 14 may also be coated with a bioingrowth material for promoting bone in-growth.

A plurality of locking washers 18 are disposed within a respective locking screw hole 26 and are configured to receive a respective locking screw 16 and fix the locking screw at a desired angle with respect to the base plate 12. Thus, the locking screws 16 lock into the base plate 12 at the junction of the locking washers 18 to create a locking screw construct. For example, the locking washers 18 may be configured to fix the locking screws 16 at an oblique angle with respect to the base plate 12, such as up to about 15 degrees in any direction with respect to the base plate. Moreover, the peg hole 28 may include a locking washer 18 positioned therein and at the base of the central peg 14 for fixing the peg locking screw 30 at a desired angle with respect to the base plate 12. Similarly, the base plate 12 may have a central hole including a locking washer 18 positioned therein for fixing the central peg 14 in position.

According to one embodiment, the locking screw holes 26 may include an annular groove 29 defined between the first 20 and second 22 surfaces for receiving a respective locking washer 18 therein, wherein a diameter and a thickness of each of the annular grooves is larger than an outer diameter and a thickness, respectively, of each of the plurality of locking washers (see FIG. 1A). Thus, the locking washers 18 may have a sufficient amount of space to “float” within the annular grooves 29 and allow for changes in angular orientation. In addition, a diameter of each of the plurality of locking screw holes 26 may be larger than an inner diameter of each of the plurality of locking washers 18 for facilitating engagement between the locking screws 16, 30 and the locking washers (see FIG. 1A). In addition, if the base plate 12 includes a curvature for conforming to the prepared bone surface as discussed above, the locking washers 18 may likewise possess such a conforming curvature.

Furthermore, the locking washers 18 may be radially resilient and configured to adjust in diameter in response to engagement with a respective locking screw 16. For instance, FIG. 1B illustrates that the locking washers 18 may have a first end 31 and a second end 33 and a slot 35 extending therebetween such that the slot allows the locking washers to possess radial resiliency. Alternatively, the locking washers 18 may comprise a resilient material. As such, the locking washers 18 are able to change their size and orientation within a respective annular groove when engaging a respective locking screw 16 due to the size and configuration of the locking screws, locking washers 18, and locking screw holes 18, as well as the resiliency of the locking washers. According to one embodiment, the locking screw and locking washer combination may be similar to that manufactured by Newclip Technics (France) and disclosed by U.S. Patent Appl. Publ. No. 2009/0318978 to Podgorski et al., which is hereby incorporated by reference in its entirety herein.

FIG. 1 illustrates that the base plate system 10 may also include a drill guide 32 that is configured to engage a locking washer 18 such that a drill bit 34 may be inserted through the drill guide and the locking screw holes 26 to drill a hole through the prepared bone surface and into the underlying bone for receiving a respective locking screw 16. Thus, the drill guide 32 may be fixed in a desired orientation and include a hole extending along its longitudinal expanse for receiving and guiding the drill bit 34 through the prepared bone surface and into the bone. The drill bit 34 may include a plurality of measurement indicators 36, such as laser etched marks, for determining a length of a respective locking screw 16 needed. Depth gauges (not shown) may also be employed to verify the depth of the drilled hole and the proper length of the locking screws 16, 30.

In another embodiment, the drill guide 32 may be configured to be engaged with each of the locking washers 18 simultaneously. Thus, the drill bit 34 may be inserted through each opening of the drill guide 32 for drilling a respective hole into the underlying bone without having to remove the drill guide after drilling each hole and reattaching the drill guide. For example, a plurality of drill guides 32 may be coupled together and configured to engage a respective locking washer 18.

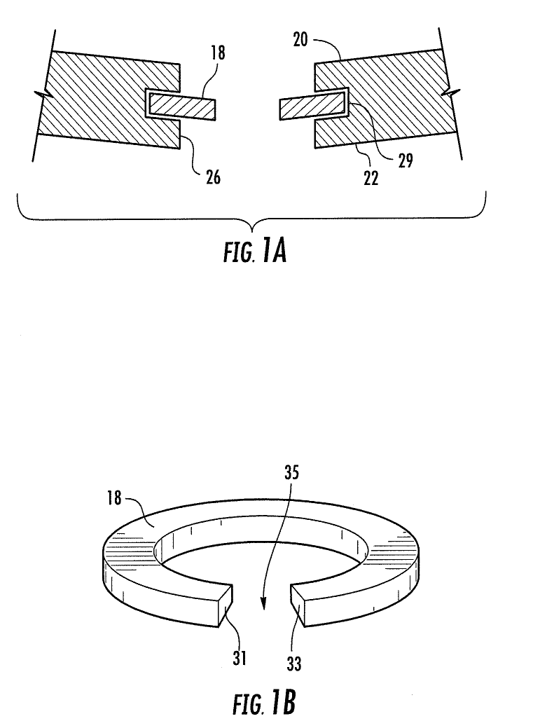

FIGS. 2 and 3 illustrate augmentation devices 40 according to additional embodiments of the present invention. The augmentation device 40 is configured to be secured to the second surface 22 of the base plate 12 and positioned adjacent to the prepared bone surface to accommodate for bone loss of the joint. Thus, the augmentation device 40 may account for bone loss such that the proper anatomical position of the base plate 12 is not compromised. The augmentation device 40 may be secured to the base plate 12 using various techniques such as fasteners or bone cement. For example, the augmentation device 40 may include a plurality of screw holes 42 configured to align with a plurality of screw holes 44 defined in the base plate 12, such that a plurality of respective screws may be inserted through the screw holes to secure the augmentation device and the base plate together. FIGS. 2 and 3 demonstrate that the base plate 12 and augmentation device 40 include a pair of screw holes 42, 44, respectively, although there may be one or more screw holes in each of the base plate and the augmentation device if desired. In addition, at least a portion of the augmentation device 40 may be coated with a bioingrowth material, as described above, or may be formed of bioingrowth material, such as material comprising Trabecular Metal™ (Zimmer Inc.).

The augmentation device 40 is generally sized and configured to align with the base plate 12. According to one aspect, the augmentation device 40 includes a pair of opposing surfaces 46, 48 and inner 50 and outer 52 edges extending therebetween, wherein a substantial portion of the outer edge corresponds to the shape of the outer surface 24 of the base plate. Thus, the augmentation device 40 may have a generally semi-circular or curved shape for conforming to a corresponding semi-circular or curved portion of the base plate 12. Moreover, in one embodiment, a pair of augmentation devices 40 may be positioned adjacent to the second surface 22 of the base plate 12 and in opposition to one another, thereby cooperating with one another to conform to the shape of the base plate and the prepared bone surface.

The augmentation device 40 may have various sizes and configurations depending on the amount of bone loss and corresponding size and configuration of the base plate 12. According to one exemplary embodiment, a maximum thickness (measured between the opposing surfaces 46, 48) of the augmentation device 40 is about 5 to 40 mm. The thickness and/or cross-sectional shape of the augmentation device 40 may be varied depending on the amount of bone loss. For instance, FIG. 2 illustrates one embodiment where the augmentation device 40 has a tapering cross-sectional shape such as a wedge cross-sectional shape, while FIG. 3 illustrates another embodiment where the augmentation device has a generally constant cross-sectional shape such as a rectangular cross-sectional shape. Thus, in one embodiment, the thickness of the augmentation device 40 may decrease from the outer edge 52 towards the inner edge 48, thus forming a wedge cross-sectional shape.

In addition, the inner edge 50 of the augmentation device 40 may include a plurality of contours 54, 56 that are configured to at least partially conform to the locking screws 16 and the central peg 14. For example, FIGS. 2 and 3 show that the contours 54, 56 have a generally semi-circular shape for extending partially around a respective locking screw 16 and central peg 14. However, it is understood that the contours 54, 56 may have different shapes according to additional aspects of the present invention, such as portions of an oval or a rectangle, and may even include holes for receiving the locking screws 16 and central peg 14. The central contour 54 configured to conform to the central peg 14 may be larger than the peripheral contours 56 that conform to the locking screws 16, although the size of the contours may vary depending on the size of the locking screws and the central peg and may be the same size if desired. As such, the contours 54, 56 are configured to be inserted between the prepared bone surface and the base plate 12 and do not interfere with the positioning of the locking screws 16 and the central peg 14. FIGS. 2 and 3 illustrate that the augmentation device 40 may include a hole 58 that is configured to align with one of the locking screw holes 26 and thereby receive a locking screw 16 therethrough. However, the augmentation device 40 may include a plurality of holes 58 configured to align with a plurality of locking screw holes 26, or the augmentation device may extend over at least one of the locking screw holes 26 in the vicinity of the screw holes 44 defined in the base plate 12. Thus, in one embodiment, the augmentation device 40 is configured to correspond to the central peg 14 and at least a pair of locking screw holes 26 positioned on opposite sides of the central peg.

The augmentation device 40 may be positioned using a system of jigs (not shown) that attach to the prepared surface of the glenoid. The jigs are configured to measure the depth of the bone loss and then allow for machining of the remaining bone to customize the fit with the augmentation device 40. The machining jig may accommodate saws and/or burrs for removing bone and to prepare the bone surface. In one embodiment, the jigs may be modified jigs used in total knee arthroplasty.

Various techniques may be employed to position the base plate system 10 and prepare the glenoid for shoulder arthroplasty, as known to those of ordinary skill in the art. For example, once the position of the base plate 12 has been determined using instrumentation such as a positioner plate having a size and configuration similar to the base plate, the glenoid surface is prepared by reaming away any remaining cartilage on the glenoid surface for receiving the bone plate 12 and any unneeded bone. The glenoid is prepared so that the second surface 22 of the bone plate 12 may lie flush against the prepared bone surface. Similarly, the position of the base plate 12 is chosen so that the base plate may be properly secured to the glenoid, while maintaining proper range of motion of the shoulder. After preparing the bone surface, the base plate 12 may then be seated on the prepared bone surface and engaged with the bone surface, such as via engagement between the central peg 14 and a peg hole drilled into the bone. In those instances where the base plate 12 is not able to be fully seated against the bone surface (e.g., due to bone loss), one or more augmentation devices 40 may be employed. In this regard, once the proper size and configuration of the augmentation device 40 is determined, the augmentation device may be positioned between the base plate 12 and the prepared bone surface and secured to the base plate, such as with fasteners or bone cement.

After the base plate 12 (or the augmentation device 40, if necessary) has been properly seated on the prepared bone surface, holes for the locking screws 16 may be drilled. For instance, holes may be drilled for receiving peripheral locking screws 16 in each of the inferior, superior, anterior, and posterior directions. In one embodiment, a drill guide 32 may be secured to one of the locking washers 18 when a desired orientation of a locking screw 16 has been determined, which may coincide with an orientation for obtaining adequate fixation and engagement with good bone. A drill bit 34 may then be inserted through the drill guide 32, and a hole may be drilled through the prepared bone surface and into the underlying bone for receiving a respective locking screw 16. This procedure for drilling each of the holes is repeated for each of the locking screws 16, including the peg locking screw 30. The depth of the holes may be verified with the measurement indicators on the drill bit 34 and/or a depth gauge (not shown) in order to determine the length of the locking screw 16 that will be necessary. The locking screws 16 and peg locking screw 30 may be inserted into a respective drilled hole and fully engaged with a locking washer 18 so as to be fixed in position. Thus, the locking screws 16 and peg locking screw 30 may be fixed to the base plate 12 in a desired orientation and extend through the prepared the bone surface and into the underlying bone. In one embodiment, the head of each locking screw 16, 30 is engaged with a respective locking washer 18 and is fully seated flush to the first surface 20 of the base plate. The locking screws 16, 30 may be inserted after each hole is drilled or after one or more of the holes have been drilled. As described above, where an augmentation device 40 is used, the contours 54, 56 are configured to at least partially conform to the locking screws 16 and the central peg 14. It is understood that the aforementioned procedure was not meant to be limiting, as various procedures and techniques may be employed to position and implant the base plate 12, as well as position and insert the locking screws 16 and the peg locking screw 30. In this regard, the procedure employed may depend on surgeon preference and the particular patient.

Therefore, embodiments of the present invention may provide several advantages. For example, the base plate system 10 may provide for improved fixation with the bone by allowing the locking screws 16, 30 to be fixed to the base plate 12 in a desired angular orientation. Moreover, the peg locking screw 30 provides additional fixation. Because the engagement between the locking screws 16, 30 and the base plate 12 via the locking washers 18 does not damage any of the components of the system 10, the locking screws may be subsequently and readily removed, such as to reposition the base plate and/or locking screws or to remove the system. The length of the central peg 14 may also be adjustable, which allows the base plate to adapt to a variety of bone structures. Furthermore, the augmentation device 40 may account for bone loss thereby allowing the base plate 12 to maintain a proper anatomical position with respect to the prepared bone surface and the humeral implant. The size and configuration of the augmentation device 40 may be customized for a particular patient to ensure that a proper fit is obtained.

Many modifications and other various embodiments of the invention set forth herein will come to mind to one skilled in the art to which this invention pertains having the benefit of the teachings presented in the foregoing descriptions and the associated drawings. Therefore, it is to be understood that the various embodiments of the invention are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A base plate system for use in a replacing a joint having a prepared bone surface, the system comprising:
a base plate comprising a plurality of locking screw holes defined therethrough and first and second opposing surfaces, at least a portion of the second surface configured to be positioned adjacent to the prepared bone surface;
a central peg extending outwardly from the second surface of the base plate and configured to at least partially engage the prepared bone surface;
a plurality of locking screws configured to be inserted within a respective locking screw hole and engage the prepared bone surface; and
a plurality of locking washers disposed within a respective locking screw hole and configured to receive a respective locking screw and lock the locking screw at a desired angle with respect to the base plate.

2. The system of claim 1, wherein each of the plurality of locking screw holes comprises an annular groove for receiving a respective locking washer therein.

3. The system of claim 2, wherein a diameter and a thickness of each of the annular grooves is larger than an outer diameter and a thickness, respectively, of each of the plurality of locking washers.

4. The system of claim 1, wherein each of the plurality of locking washers is radially resilient and configured to adjust in diameter in response to engagement with a respective locking screw.

5. The system of claim 1, wherein a diameter of each of the plurality of locking screw holes is larger than an inner diameter of each of the plurality of locking washers.

6. The system of claim 1, wherein the central peg is integrally formed with the base plate.

7. The system of claim 6, further comprising a peg locking screw integrally formed with the central peg and the base plate.

8. The system of claim 1, wherein the central peg is configured to be secured to the base plate and is variable in length.

9. The system of claim 1, wherein the central peg comprises a peg hole configured to receive a peg locking screw therethrough for engaging the bone.

10. The system of claim 9, further comprising a locking washer positioned within the peg hole and configured to lock the peg locking screw at a desired angle with respect to the base plate.

11. The system of claim 9, wherein the peg locking screw has a length of about 10 to 45 mm and a diameter of about 6 to 7 mm.

12. The system of claim 1, wherein at least a portion of the second surface of the base plate and the central peg are coated with a bioingrowth material.

13. The system of claim 1, wherein the central peg has a length of about 10 to 40 mm.

14. The system of claim 1, wherein each of the plurality of locking screws has a length of about 5 to 45 mm and a diameter of about 4 to 6 mm.

15. The system of claim 1, wherein the plurality of locking washers are configured to lock the locking screw at an angle of up to about 15 degrees in any direction with respect to the base plate.

16. The system of claim 1, further comprising a drill guide configured to engage one of the plurality of locking washers and a drill bit configured to be inserted through the drill guide and drill a hole through the prepared bone surface for receiving a respective locking screw.

17. The system of claim 1, further comprising an augmentation device configured to be secured to the base plate and positioned adjacent to the prepared bone surface to accommodate for bone loss of the joint.

18. The system of claim 17, wherein the augmentation device comprises a plurality of screw holes configured to align with a plurality of screw holes defined in the base plate, and wherein the augmentation device is configured to be secured to the base plate by inserting a plurality of respective screws through the screw holes defined in the augmentation device and the base plate.

19. The system of claim 17, wherein the augmentation device has a wedge or rectangular cross-sectional shape.

20. The system of claim 17, wherein a maximum thickness of the augmentation device is about 5 to 40 mm.

21. The system of claim 17, wherein at least a portion of the augmentation device is coated with, or formed of, a bioingrowth material.

22. The system of claim 17, wherein the augmentation device comprises a pair of opposing surfaces and an inner edge and an outer edge extending therebetween, and wherein a substantial portion of the outer edge corresponds to an outer edge of the base plate.

23. The system of claim 22, wherein the inner edge of the augmentation device comprises a plurality of contours configured to at least partially conform to the plurality of locking screws and the central peg.

24. The system of claim 17, wherein the augmentation device comprises at least one hole configured to align with a respective one of the plurality of locking screw holes and thereby receive a locking screw therethrough.

25. The system of claim 1, wherein the base plate is configured to be positioned adjacent to a prepared bone surface of the glenoid and secured thereto with the plurality of locking screws.

26. A method for installing a bone plate system for replacing a joint having a prepared bone surface, the method comprising:
positioning a base plate adjacent to the prepared bone surface such that a central peg extending outwardly from the base plate engages the prepared bone surface, the base plate comprising a plurality of locking screw holes defined therethrough and a plurality of locking washers disposed within a respective locking screw hole; and
inserting a plurality of locking screws through a respective locking screw hole and locking washer such that the locking screws are fixed therein and extend through the prepared bone surface at a desired angle with respect to the base plate.

27. The method of claim 26, wherein inserting comprises inserting the plurality of locking screws through a respective locking screw hole and locking washer such that the locking screws are fixed at an angle of up to about 15 degrees in any direction with respect to the base plate.

28. The method of claim 26, further comprising inserting a peg locking screw through a peg hole defined in the central peg such that the peg locking screw engages the prepared bone surface.

29. The method of claim 28, wherein inserting the peg locking screw comprises inserting the peg locking screw through a locking washer positioned within the peg hole to lock the peg locking screw at a desired angle with respect to the base plate.

30. The method of claim 26, further comprising adjusting a length of the central peg.

31. The method of claim 26, further comprising attaching a drill guide to one of the plurality of locking washers and inserting a drill bit through the drill guide to drill a hole through the prepared bone surface for receiving a respective locking screw.

32. The method of claim 26, further comprising securing an augmentation device to the base plate and positioning the augmentation device adjacent to the prepared bone surface to accommodate for bone loss of the joint.

33. The method of claim 32, wherein securing comprising positioning a plurality of contours defined in the augmentation device at least partially around the locking screws and the central peg.

34. The method of claim 26, wherein positioning comprises positioning the base plate adjacent to a prepared bone surface of the glenoid.
